(19) United States
(12) Patent Application Publication
(10) Pub. No.: US 2015/0196366 A1
(43) Pub. Date: Jul. 16, 2015

(54) DEVICE FOR POSITIONING JOINT LINE FOR REVISION TOTAL KNEE ARTHROPLASTY

(71) Applicant: CORENTEC CO., LTD., Cheonan-si (KR)

(72) Inventors: Seok-Joo Kim, Gwangju-si (KR); Jun-kyu Park, Cheonan-si (KR); Jung-Sung Kim, Seoul (KR); Chil-Hong Moon, Seoul (KR)

(21) Appl. No.: 14/595,906

(22) Filed: Jan. 13, 2015

(30) Foreign Application Priority Data
Jan. 14, 2014 (KR) 10-2014-0004592

(51) Int. Cl. A61B 19/00

(52) U.S. Cl. CPC A61B 19/46; A61B 2019/467

(57) ABSTRACT

A device for positioning a knee joint line for revision total knee arthroplasty. In revision total knee arthroplasty, the position of the joint line of an existing femoral element is measured before the existing femoral element is removed from the femur in revision total knee arthroplasty. A new femoral element is implanted according to the measured position of the joint line. The joint line is formed at the same position as the joint line of the other leg. The position of the surface of the femur to be cut is determined based on the position of the joint line when a loss or the like in the femur must be compensated for by implanting a block between the femur and the femoral element.

### CROSS-REFERENCE TO RELATED APPLICATIONS

This application claims priority to Korean Application No. 10-2014-0004592, filed Jan. 14, 2014, which is incorporated herein by specific reference.

### BACKGROUND OF THE INVENTION

1. Field of the Invention

The present invention relates to a device for positioning a knee joint line for revision total knee arthroplasty. More particularly, the present invention relates to a device for positioning a knee joint line for revision total knee arthroplasty, in which, in the case of revision total knee arthroplasty, the position of the joint line of an existing femoral element is measured before the existing femoral element is removed from the femur in revision total knee arthroplasty, and a new femoral element can be implanted according to the measured position of the joint line, such that the joint line is formed at the same position as the joint line of the other leg, and such that the position of the surface of the femur to be cut can be determined based on the position of the joint line when a loss or the like in the femur must be compensated for by implanting a block between the femur and the femoral element.

2. Description of the Related Art

Among a number of joints in the human body, the knee joint refers to a joint connecting the tibia and the femur. The knee joint is positioned between the lower end of the femur, the upper end of the tibia and the backside of the patella (a bone in front of the knee that protects the knee joint), and allows the leg to bend backward at the knee. Recently, the number of patients whose knee joint is irrecoverable due to the wearing of the knee joint, the aging of bone tissues or an accident is increasing. When the knee joint has lost its function due to the arthritis or a wound, artificial joint arthroplasty is performed to transplant an implant so that an artificial joint can substitute for the damaged knee joint to properly function.

A description will be given below of an artificial knee joint applied to artificial joint replacement with reference to Patent Document below and FIG. 1. The artificial knee joint includes a femoral element 400 coupled with a femur 100 and a tibia element 500 coupled with a tibia 200. The femoral element 400 reproduces the actual motion of a knee along a contact surface on which the femoral element 400 adjoins the tibia element 500. However, when the artificial joint has worn or has lost its function due to a damaged bone or ligament, revision total knee arthroplasty must be performed, in which the existing artificial knee joint is removed and is replaced with a new artificial knee joint. In the revision total knee arthroplasty, a new femoral element and a new tibia element are fixed to the bones through a series of surgical operations including: removing the existing femoral element 400 and the tibia element 500 from the femur 100 and the tibia 200; setting the positions and sizes of the new femur and tibia elements; and forming new cut surfaces of the bones. When the new femoral element is implanted to the femur, the new femoral element must be disposed such that the knee bends at the same position as the knee of the other leg. The portion of the femoral element that protrudes most toward the tibia adjoins the upper surface of the tibia. A line defined by the portion where the femoral element adjoins the tibia element in the state in which the knee is completely stretched is referred to as a joint line L, along which the artificial knee joint bends.

Therefore, the depth to which the femoral element is implanted into the femur must be determined according to the position of the joint line L. In the related art, the position of the joint line L was determined based on preset indexes, such as 3 cm from the medial epicondyle 101 of the femur toward the tibia, 2.5 cm from the lateral epicondyle 103 toward the tibia and 2 cm from the bottom 301 of the patella 300 toward the tibia. It is difficult to determine the position of the joint line L when part of the bone in either the medial epicondyle 101 or the lateral epicondyle 103 is lost or the position of the patella 300 is changed. In such a case, the position of the joint line must be determined by reviewing an X-ray picture that was taken before or visually comparing with the other leg, thereby increasing the operation time. In addition, the position of the joint line may not be precisely determined, and thus both knees may bend at different positions. This makes it impossible for the patient to have a proper gait, and the patient's weight is biased to one leg, thereby increasing the load of the knee joint.

The information disclosed in the Background of the Invention section is only for the enhancement of understanding of the background of the invention, and should not be taken as an acknowledgment or as any form of suggestion that this information forms a prior art that would already be known to a person skilled in the art.

### RELATED ART DOCUMENT

Patent Document 1: Korean Patent No. 10-0901524 (Patented Jun. 1, 2009), titled “ARTIFICIAL KNEE JOINT IN WHICH A PLURALITY OF CURVATURE PARTS ARE FORMED IN FEMUR COUPLING MEMBER”

### SUMMARY OF THE INVENTION

Accordingly, the present invention has been made keeping in mind the above problems occurring in the related art, and the present invention is intended to propose a device for positioning a joint line for revision total knee arthroplasty, in which, in the case of revision total knee arthroplasty, the position of the joint line of an existing femoral element is measured before the existing femoral element is removed from the femur in revision total knee arthroplasty, and a new femoral element can be implanted according to the measured position of the joint line, such that the joint line is formed at the same position as the joint line of the other leg.

The present invention is also intended to propose a device for positioning a joint line for revision total knee arthroplasty, in which, in the case of revision total knee arthroplasty, the position of the joint line of an existing femoral element is measured before the existing femoral element is removed from the femur in revision total knee arthroplasty, such that the position of the surface of the femur to be cut can be determined based on the position of the joint line when a loss or the like in the femur must be compensated for by implanting a block between the femur and the femoral element.

The present invention is also intended to propose a device for positioning a joint line for revision total knee arthroplasty. The device includes a support member fixedly fitted into a predetermined portion of a femur and a joint line measuring part supported by the support member to measure the position of a joint line. It is possible to easily measure the position of the joint line of the existing femoral element and implant a new femoral element to a correct position while checking the position of the joint line.

The present invention is also intended to propose a device for positioning a joint line for revision total knee arthroplasty, in which a hole is formed in one side of the joint line measuring part. Since the joint line measuring part can be fitted around the support member, the measuring operation can be finished in a short time. When a new femoral element is to be implanted, it is possible to determine the position where the new femoral element is to be implanted by fitting the joint line measuring part around the support member. The process of determining the position of the joint line can be easily and precisely carried out.

The present invention is also intended to propose a device for positioning a joint line for revision total knee arthroplasty. The device includes an indicator consisting of markings formed at fixed intervals on one surface of one side of the joint line measuring part. The indicator is formed as scales or length-indicating numbers, allowing the position of the joint line to be precisely measured. When a new femoral element is implanted, the joint line can be formed at a correct position.

The present invention is also intended to propose a device for positioning a joint line for revision total knee arthroplasty, in which a bent position adjusting part protrudes from one portion of the joint line measuring part. The position adjusting part has a second hole which the support member can extend through and be fitted into, such that the support member can be selectively fitted into the hole or the second hole. It is therefore possible to adjust the position of the joint line measuring part according to the surgical environment.

The present invention is also intended to propose a device for positioning a joint line for revision total knee arthroplasty. The device includes an ML size measuring part. One side of the ML size measuring part is fitted around the joint line measuring part, and scales or numbers functioning as a reference for the measurement of lengths are marked on one surface of the other side of the ML size measuring part. It is therefore possible to easily determine the size of the new femoral element.

In order to achieve the above object, the present invention is realized by following embodiments.

According to one aspect of the present invention, there is provided a device for positioning a joint line for revision total knee arthroplasty, in which a knee bends along the joint line. The device includes: a support member fixedly fitted into a predetermined portion of a femur; and a joint line measuring part supported by the support member, wherein the joint line measuring part measures a position of a joint line formed on a distal end of a femoral element to be coupled with the femur, whereby the position of the joint line is to be easily determined when a new femoral element is implanted.

According to an embodiment of the present invention, in the device for positioning a joint line for revision total knee arthroplasty, the joint line measuring part may include an indicator including markings disposed at fixed intervals on one surface of one side of the joint line measuring part, the indicator allowing the position of the joint line to be measured.

According to another embodiment of the present invention, in the device for positioning a joint line for revision total knee arthroplasty, the joint line measuring part may have a hole formed in one side thereof, the support member being to be fitted into the hole of the joint line measuring part.

According to a further embodiment of the present invention, in the device for positioning a joint line for revision total knee arthroplasty, the joint line measuring part may include a bent position adjusting part protruding from one portion of the joint line measuring part, the position adjusting part having a second hole which the support member is to extend through and be fitted into, such that the position of the joint line measuring part is adjustable.

According to another embodiment of the present invention, in the device for positioning a joint line for revision total knee arthroplasty, the device may further include an ML size measuring part coupling with the joint line measuring part to measure an ML size of the femoral element.

According to a further embodiment of the present invention, in the device for positioning a joint line for revision total knee arthroplasty, the ML size measuring part may include: a holder formed in one side the ML size measuring part in a penetrating fashion such that one side of the joint line measuring part is fitted into the holder; and an indicator including markings disposed at fixed intervals on one surface of the other side of the ML size measuring part, the indicator allowing a lateral size of the femoral element to be measured.

The present invention can have the following effects that will be apparent from the combination and relationship between the above-mentioned embodiments and the following Detailed Description of the Invention.

According to the present invention, in the case of revision total knee arthroplasty, the position of the joint line of an existing femoral element is measured before the existing femoral element is removed from the femur in revision total knee arthroplasty, and a new femoral element can be implanted according to the measured position of the joint line, such that the joint line is formed at the same position as the joint line of the other leg.

According to the present invention, in the case of revision total knee arthroplasty, the position of the joint line of an existing femoral element is measured before the existing femoral element is removed from the femur in revision total knee arthroplasty, such that the position of the surface of the femur to be cut can be determined based on the position of the joint line when a loss or the like in the femur must be compensated for by implanting a block between the femur and the femoral element.

According to the present invention, the device includes a support member fixedly fitted into a predetermined portion of a femur and a joint line measuring part supported by the support member to measure the position of a joint line. It is possible to easily measure the position of the joint line of the existing femoral element and implant a new femoral element to a correct position while checking the position of the joint line.

According to the present invention, a hole is formed in one side of the joint line measuring part. Since the joint line measuring part can be fitted around the support member, the measuring operation can be finished in a short time. When a new femoral element is to be implanted, it is possible to determine the position where the new femoral element is to be implanted by fitting the joint line measuring part around the support member. The process of determining the position of the joint line can be easily and precisely carried out.

According to the present invention, the device includes an indicator consisting of markings formed at fixed intervals on one surface of one side of the joint line measuring part. The indicator is formed as scales or length-indicating numbers, allowing the position of the joint line to be precisely measured. When a new femoral element is implanted, the joint line can be formed at a correct position.

According to the present invention, a bent position adjusting part protrudes from one portion of the joint line measuring part. The position adjusting part has a second hole that the support member can extend through and be fitted into, such that the support member can be selectively fitted into the hole or the second hole. It is therefore possible to adjust the position of the joint line measuring part according to the surgical environment.

According to the present invention, the device includes an ML size measuring part. One side of the ML size measuring part is fitted around the joint line measuring part, and scales or numbers functioning as a reference for the measurement of lengths are marked on one surface of the other side of the ML size measuring part. It is therefore possible to easily determine the size of the new femoral element.

### BRIEF DESCRIPTION OF THE DRAWINGS

The above and other objects, features and advantages of the present invention will be more clearly understood from the following detailed description when taken in conjunction with the accompanying drawings, in which:

FIG. 1 is a reference view illustrating a joint line positioning indicator of an artificial knee joint of the related art;

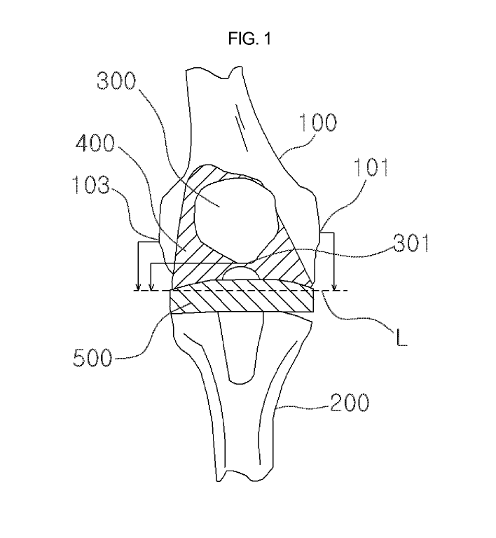

FIG. 2 is a perspective view illustrating a device for positioning a joint line for revision total knee arthroplasty according to an exemplary embodiment of the invention;

FIG. 3 is a side elevation view of the device for positioning a joint line shown in FIG. 2;

FIG. 4 is an end view of the joint line measuring part shown in FIG. 2;

FIG. 5 is an end view illustrating another embodiment of the joint line measuring part shown in FIG. 2;

FIG. 6 is a perspective view illustrating a device for positioning a joint line for revision total knee arthroplasty according to another exemplary embodiment of the invention;

FIG. 7 is a front elevation view of the device for positioning a joint line shown in FIG. 6;

FIG. 8 is a reference view for explaining the process of positioning a surface of a bone to be cut using the device for positioning a joint line shown in FIG. 2.

### DETAILED DESCRIPTION OF THE INVENTION

Reference will now be made in greater detail to a device for positioning a joint line for revision total knee arthroplasty according to the present invention in conjunction with the accompanying drawings. Unless otherwise defined, all terms used herein have the same meaning as commonly understood by a person skilled in the art to which the present invention belongs. If the meanings of the terms are not consistent, the terms will be interpreted as being defined herein. In the following description of the present invention, detailed descriptions of known functions and components incorporated herein will be omitted in the case that the subject matter of the present invention is rendered unclear.

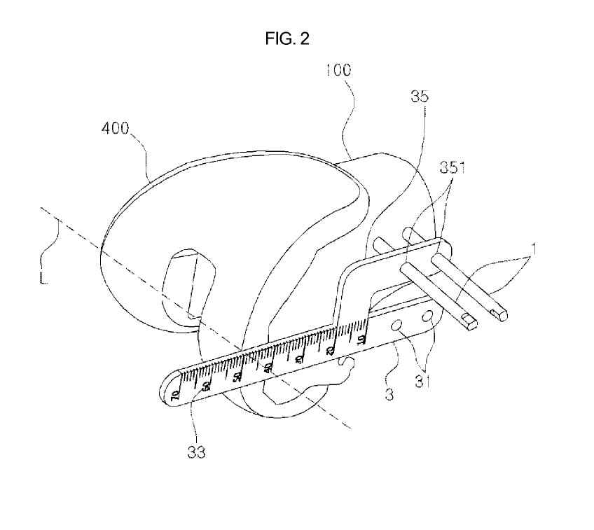

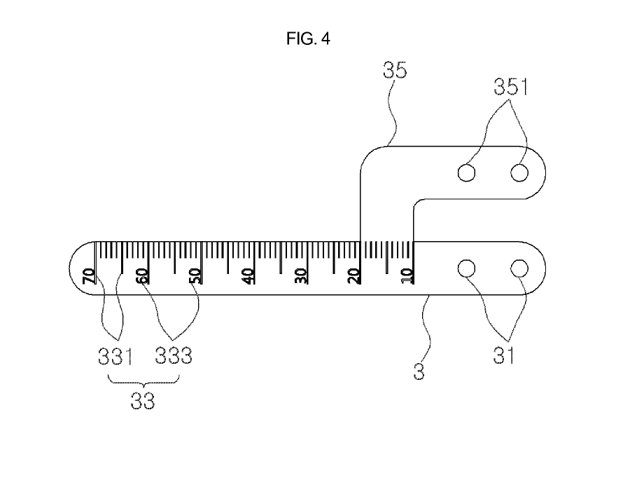

The device for positioning a joint line for revision total knee arthroplasty according to an exemplary embodiment of the present invention will be described with reference to FIGS. 2 to 8. The device for positioning a joint line includes a support members 1 fixedly fitted into predetermined portions of a femur 100 and a joint line measuring part 3 for measuring the position of a joint line L formed at the distal end of a femoral element 400 that is supported on the support members 1 and is coupled with the femur 100. The device for positioning a joint line facilitates the positioning of the joint line L when a new femoral element is inserted.

The device for positioning a joint line according to this embodiment is used for revision total knee arthroplasty in which an implanted existing artificial knee joint is removed and a new artificial knee joint is implanted, and is used for determining a joint line L where the knee is bent. As described above, the joint line L indicates a line connecting the distal portions of the femoral element adjacent to the tibia, i.e. the portions of the femoral element that most protrudes toward the tibia, and the knee bends along the joint line L. It is, of course, required for a human that the joint lines L where both knees bend are formed at the same position. If the joint lines L are not at the same position, a normal gait or running is impossible, and weight is biased to the knee joint on one side, thereby causing more severe damage to the knee joint. Therefore, when the artificial knee joint is implanted, it is required that the joint lines L of both legs be formed at the same position. Considering that the joint lines L of both legs were formed at the same position in total knee arthroplasty in which an artificial knee joint was initially implanted, it is possible to easily determine the position of the joint line L if the position of the joint line L of the existing femoral element 400 can be measured and the position of the joint line L that was measured using the existing femoral element 400 can be used when implanting the new femoral element into the femur. This is one of the objects of the invention, and hereinafter, the configuration of the invention will be described focusing on this object.

As illustrated in FIG. 2, the support members 1 are fixedly fitted into predetermined portions of the femur 100, sets a reference point with which the position of the joint line L is to be measured and determined, and are to be fitted into holes 31 of the joint line measuring part 3, which will be described later. In other words, the support members 1 are fixed fitted into predetermined portions of the femur 100 (preferably, inner or outer portions of the femur; herein, the front and back sides of the knee will be referred to as the front and rear sides, and the inner and outer sides of the knee will be referred to as the inner and outer sides), and the position of the joint line L is measured by fitting the joint line measuring part 3 to be described later around the fixed support members 1. After the position of the joint line L indicated by the joint line measuring part 3 is recorded, when a new femoral element is to be implanted into the femur, the new femoral element is implanted according to the position of the initially-measured position by fixedly fitting the support members 1 again into the initially fitted portions and fitting the joint line measuring part 3 around the support members 1. In this manner, the joint line L can be formed at the same position as the existing joint line L. Therefore, the support members 1 can be fitted into any portions, more particularly, into portions where the position of the joint line L is easily measured according to several environmental factors such as the geometry of either the femur or the joint line measuring part 3. Although the support members 1 are preferably in the shape of elongated pins such that the support members 1 can be fixedly fitted into the femur 100, a variety of other means able to support the joint line measuring part 3 may be applied. In addition, the support members 1 may be provided in a plurality of numbers in order to reliably fix the joint line measuring part 3, and according to this embodiment, two support members 1 are fixedly fitted into the femur. However, the number and array of the support members 1 may vary according to the holes 31 of the joint line measuring part 3, which will be described later.

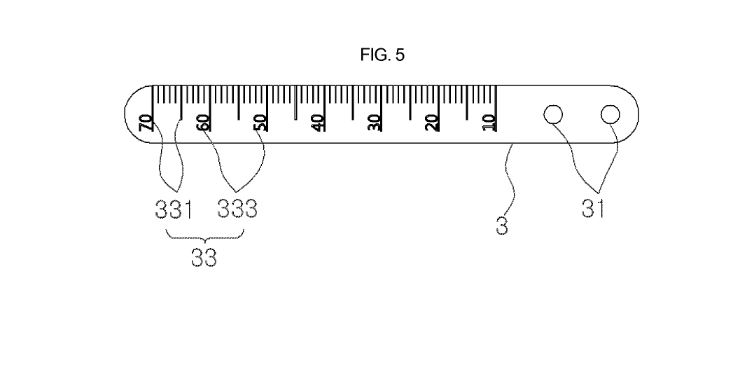

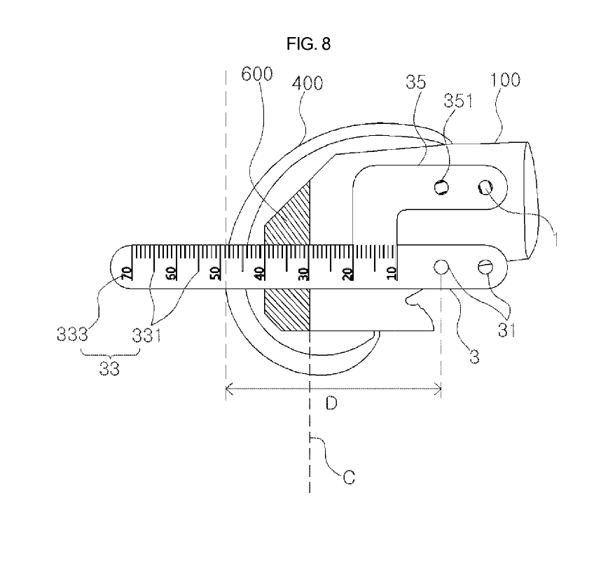

As illustrated in FIG. 2, the joint line measuring part 3 is supported by the support members 1, and serves to measure the position of the joint line L. When a new femoral element is implanted, the joint line measuring part 3 allows the new femoral element to be implanted according to the position of the measured joint line L. It is preferable that the joint line measuring part 3 is in the shape of a thin plate extending a predetermined length. The joint line measuring part 3 may have any shape as long as it can measure the position of the joint line L while being supported by the support members 1. Therefore, the joint line measuring part 3 may further include a position adjusting part 35 that allows the position of the joint line measuring part 3 to be adjusted. As illustrated in FIG. 5, the joint line measuring part 3 may be in the shape of a plate extending a predetermined length without the position adjusting part 35. In addition, when a block 600 is implanted between the femur 100 and the femoral element 400 because the femur 100 is damaged, as illustrated in FIG. 8, the joint line measuring part 3 can determine the position C of the surface of the femur 100 to be cut into which the block 600 is to be fitted based on the measured position of the joint line L. In other words, when the femur 100 is damaged, the damage in the bone must be compensated by implanting the block 600 according to a predetermined standard. In this case, the femur 100 must be cut at a suitable position such that the block 600 can be reliably implanted at a correct position. Since the joint line L must be formed at the same position, the position C of the surface of the bone to be cut can be determined such that the same joint line L is formed in consideration of the position of the joint line L measured by the joint line measuring part 3 and the thickness of the block 600. The joint line measuring part 3 has the holes 31 formed in one side and an indicator 33 on the other side. The indicator 33 consisting of markings at fixed intervals on one surface of the other side of the joint line measuring part 3 functions as a reference in the measurement of the position of the joint line L. The joint line measuring part 3 also includes the position adjusting part 35 that protrudes at a predetermined point and subsequently is bent, allowing the position of the joint line measuring part 3 to be adjusted.

The holes 31 are perforated in one side of the joint line measuring part 3 such that the support members 1 can be fitted into the holes 31 to support the joint line measuring part 3. The size of the holes 31 corresponds to the diameter of the support members 1. When a plurality of the support members 1 are provided, a corresponding number of the holes 31 may be provided. In addition, as described above, the number and array of the holes 31 may be variously provided corresponding to the number and array of the support members 1 such that the support members 1 can be fitted into the holes 31. It is also apparent that the holes 31 may be variously arranged according to the shape of the joint line measuring part 3.

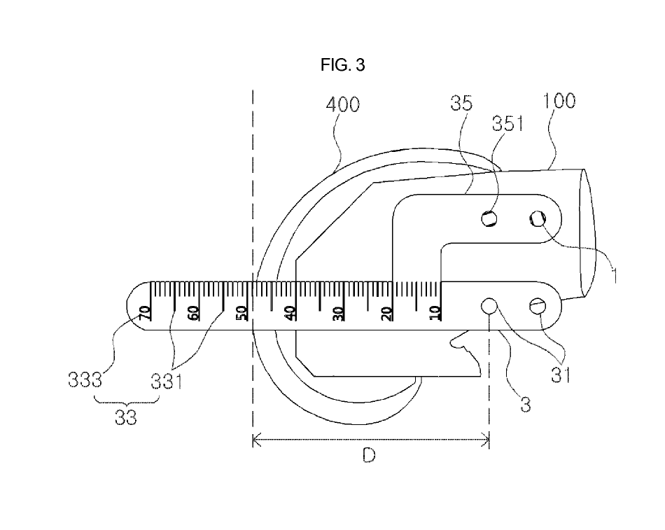

As illustrated in FIG. 3, the indicator 33 is a configuration that is provided on one surface of the other side of the joint line measuring part 3, functioning as a reference in the measurement of the position of the joint line L. The indicator 33 includes line marks 331 at fixed intervals and number marks 333, allowing the position of the joint line L to be measured and recorded. With the indicator 33, when a new femoral element is implanted into the femur 100, the new femoral element can be implanted according to the measured position of the joint line L.

The line marks 331 are scales marked at fixed intervals, allowing the position of the joint line L to be recognized. It is preferable that the indicator 33 extends a sufficient length such that the joint line measuring part 3 can be used to measure various femoral elements having a variety of sizes as well as the position of the joint line L. It is preferable that the line marks 331 are marked at an every interval of 1 mm and longer line marks are marked at an every interval of 5 mm such that a measured position is easily recognizable. However, this is not intended to be limiting and the line marks may be provided at a variety of intervals and sizes.

The number marks 333 are numbers marked corresponding to the line marks 331, allowing the position of the joint line L to be recognized and recorded. As illustrated in FIG. 3, it is preferable that the distance D from the holes 31 to the joint line L is marked at intervals of 10 mm. However, since the invention is intended to measure and record the position of the joint line L of the existing femoral element 400 and allow the same joint line L to be formed when the new femoral element 400 is implanted, it is not necessarily required that the number marks 333 indicate the distance from the holes 31 to the joint line L. Rather, any types of numbers allowing the position to be recognized and recorded may be used as the number marks.

The position adjusting part 35 protrudes upward from a predetermined point of the joint line measuring part 3, and is perpendicularly bent backward to be parallel to the joint line measuring part 3. The position adjusting part 35 has second holes 351 that the support members 1 can pass through and be fitted into, allowing the joint line measuring part 3 to be adjusted in position. It is preferable that a plurality of the second holes 351 is provided like the holes 31. The size of the second holes 351 corresponds to the diameter of the support members 1. The number and array of the second holes 351 may be variously provided corresponding to the number and array of the support members 1 such that the support members 1 can be fitted into the holes 351. The second holes 351 may be provided in a variety of arrays according to the shape of the position adjusting part 35. The position adjusting part 35 controls the position of the joint line measuring part 3 by adjusting the positions where the support members 1 are fitted. The position adjusting part 35 allows the support members 1 to be selectively fitted into the holes 31 or the second holes 351 according to the geometry of the femur 100 or the femoral element 400 or the surgical environment. Accordingly, the support members 1 can be fitted into and reliably fixed to suitable positions of the femur 100, and at the same time, the position of the joint line L can be precisely measured at the optimum position.

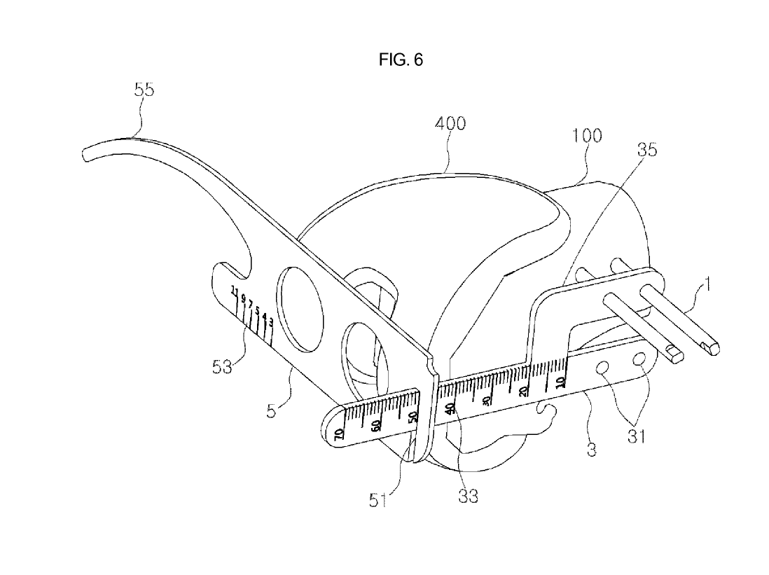

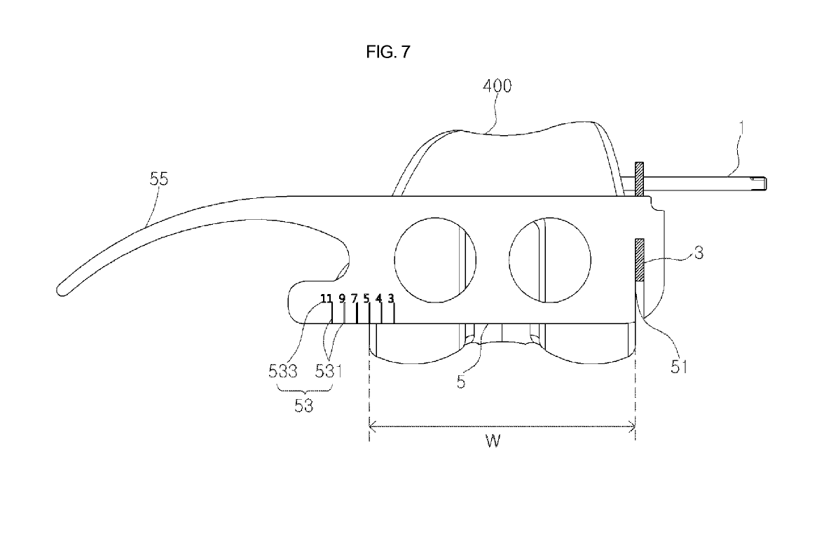

As illustrated in FIGS. 6 and 7, the device for positioning a joint line according to the invention includes an ML size measuring part 5 for measuring the ML size W of the femoral element 400. The ML size W of the femoral element 400 refers to the length of a straight line connecting the portion of the femoral element 400 that protrudes most inward and the portion of the femoral element 400 that protrudes most outward. It is possible to determine the size of the new femoral element to be implanted by measuring the ML size W of the femoral element 400.

The ML size measuring part 5 includes a holder 51, an indicator 53 and a gripping portion 55. The holder 51 is formed by perforating one side of the ML size measuring part 5 such that the other side of the joint line measuring part 3 is fitted into the holder 51. The indicator 53 is configured as markings formed on one surface of the other side of the ML size measuring part 5 at fixed intervals, allowing the ML size W of the femoral element 400 to be measured. The gripping portion 55 allows the ML size measuring part 5 to be easily held. The ML size measuring part 5 is fitted into the other side of the joint line measuring part 3 in the state in which the joint line measuring part 3 is in close contact with the femoral element 400. The ML size measuring part 5 measures the ML size W of the femoral element 400 in close contact with the femoral element 400.

The holder 51 penetrates through one side of the ML size measuring part 5 such that the joint line measuring part 3 can be fitted into the holder 51. It is preferable that the holder 51 is opened downward in order to facilitate the fitting of the joint line measuring part 3 having a variety of width. The joint line measuring part 3 is fitted into the holder 51 in the state in which the joint line measuring part 3 closely adjoins one side of the femoral element 400. Accordingly, the holder 51 is positioned at the inner portion that protrudes most inward or the outer portion that protrudes most outward, thereby forming a one-side reference point of the ML size W of the femoral element 400.

The indicator 53 is a configuration that is formed on one surface of the other side of the ML size measuring part 5, functioning as a reference for the measurement of the ML size W of the femoral element 400. The indicator 53 includes line marks 531 at fixed intervals and number marks 533, allowing the ML size W of the femoral element 400 to be measured. The indicator 53 may also be referred to when a new femoral element is to be determined.

The line marks 531 are scales marked at fixed intervals, allowing the position of the most-protruding inner or outer portion of the femoral element 400 opposite the holder 51 to be recognized. It is preferable that the indicator 53 extends a sufficient length such that the ML size W of the femoral element 400 having a variety of sizes can be measured. In other words, when the holder 51 is positioned on the portion of the femoral element 400 that protrudes most inward, the line marks 531 are used to measure the portion of the femoral element 400 that protrudes most outward. When the holder 51 is positioned on the portion of the femoral element 400 that protrudes most outward, the line marks 531 are used to measure the portion of the femoral element 400 that protrudes most inward. In this manner, the ML size W of the femoral element 400 is measured. The line marks 531 may have a variety of intervals and sizes as long as the size of a newly-implanted femoral element can be determined.

The number marks 533 are numbers marked corresponding to the line marks 531, allowing the ML size W of the femoral element 400 to be recognized and the size of a corresponding new femoral element to be determined. Although the actual length indicating the ML size W of the femoral element 400 may be marked, this is not intended to be limiting. As illustrated in FIG. 7, the standards or the selectable numbers of femoral elements corresponding to the positions of the line marks 531 may be marked. Any marking system is applicable as long as this marking system can measure the ML size W of the femoral element 400 and determine the size of a new femoral element.

The gripping portion 55 protrudes from the other side of the ML size measuring part 5 to hold the ML size measuring part 5, in which the ML size W of the femoral element is easily recognizable through the indicator 53 even in the state in which the ML size measuring part 5 is in close contact with the femoral element 400. Although it is preferable that the gripping portion 55 is in the shape of a handle that can be held with the hand, the gripping portion 55 may have any shape as long as a user can easily measure the ML size W of the femoral element while holding the ML size measuring part 5.

A description will be given below of the process of the use of the device for positioning a joint line with reference to FIGS. 2 to 8. Before the existing femoral element 400 is removed from the femur 100, the support members 1 are fitted into and fixed to predetermined positions of the femur 100, and the joint line measuring part 3 is brought into close contact with one side of the femoral element 400 by fitting the holes 31 or the second holes 351 around the support members 1. In the state in which the joint line measuring part 3 is in close contact with one side of the femoral element 400, the position of the portion of the femoral element 400 that protrudes most toward the femur is measured using the indicator 33 and the measured position is separately recorded. This position is the position of the joint line L. In addition, the other side of the joint line measuring part 3 is fitted into the holder 51 of the ML size measuring part 5, and the ML size measuring part 5 is brought into close contact with the femoral element 400 in the state in which the gripping portion 55 is held. At this point, the position of the portion positioned opposite the holder 51, i.e. the portion that most protrudes toward one side of the femoral element 400, is measured through the indicator 53 of the ML size measuring part 5. This position is the ML size W of the femoral element 400, and is referred to in the determination of the size of a new femoral element. When the measurement on the position of the joint line L and the ML size W of the femoral element 400 is completed, the existing femoral element 400 is removed from the femur 100, and a series of surgical operations of, for example, setting the position and size of a new femoral element to be implanted and forming a cut surface of the bone is undertaken. After the series of surgical operations, when the new femoral element is prepared, the new femoral element is implanted into the femur 100. Here, the support members 1 are fitted into the initial position again and are fixed thereto, the joint line measuring part 3 is fitted around the support members 1, and subsequently the new femoral element is inserted by a predetermined distance with reference to the recorded position of the joint line L such that the same joint line L is formed. Accordingly, the joint line L is formed at the same position as the previous joint line such that the joint line L is at the same position as the joint line of the other leg.

In addition, as described above, when the block 600 has to be disposed between the femur 100 and the femoral element due to a damage in the femur 100, it is possible to dispose the support members 1 and the joint line measuring part 3 in the process of forming the cut surface of the bone and cut the femur 100 in the correct position C considering the position of the joint line L and the thickness of the block 600.

Although the exemplary embodiments of the present invention have been described for illustrative purposes, a person skilled in the art will appreciate that various modifications, additions and substitutions are possible, without departing from the scope and spirit of the present invention as disclosed in the accompanying claims.

What is claimed is:

1. A device for positioning a joint line for revision total knee arthroplasty, in which a knee bends along the joint line, the device comprising:
a support member fixedly fitted into a predetermined portion of a femur; and
a joint line measuring part supported by the support member, wherein the joint line measuring part measures a position of a joint line formed on a distal end of a femoral element to be coupled with the femur,
whereby the position of the joint line is to be easily determined when a new femoral element is implanted.

2. The device according to claim 1, wherein the joint line measuring part comprises an indicator including markings disposed at fixed intervals on one surface of one side of the joint line measuring part, the indicator allowing the position of the joint line to be measured.

3. The device according to claim 2, wherein the joint line measuring part has a hole formed in one side thereof, the support member being to be fitted into the hole of the joint line measuring part.

4. The device according to claim 1, wherein the joint line measuring part comprises a bent position adjusting part protruding from one portion of the joint line measuring part, the position adjusting part having a second hole which the support member is to extend through and be fitted into, such that the position of the joint line measuring part is adjustable.

5. The device according to claim 1, further comprising an ML size measuring part coupling with the joint line measuring part to measure an ML size of the femoral element.

6. The device according to claim 5, wherein the ML size measuring part comprises:
a holder formed in one side the ML size measuring part in a penetrating fashion such that
one side of the joint line measuring part is fitted into the holder; and
an indicator including markings disposed at fixed intervals on one surface of the other side of the ML size measuring part, the indicator allowing a lateral size of the femoral element to be measured.

7. The device according to claim 2, further comprising an ML size measuring part coupling with the joint line measuring part to measure an ML size of the femoral element.

8. The device according to claim 3, further comprising an ML size measuring part coupling with the joint line measuring part to measure an ML size of the femoral element.

9. The device according to claim 4, further comprising an ML size measuring part coupling with the joint line measuring part to measure an ML size of the femoral element.
